(19) United States
(12) Patent Application Publication
(10) Pub. No.: US 2015/0086606 A1
(43) Pub. Date: Mar. 26, 2015

(54) BIORESORBABLE AND BIOACTIVE THREE-DIMENSIONAL POROUS MATERIAL AND METHOD FOR THE PRODUCTION THEREOF

(71) Applicant: BIOACTIVE TECNOLOGIA EM POLIMEROS LTDA-ME, São Poulo - SP (BR)

(72) Inventors: Walter Israel Rojas Cabrera, Sao Paulo - SP (BR); Karina Nakajima, Sao Paulo - SP (BR); Luana Bendo, Sao Paulo - SP (BR)

(21) Appl. No.: 14/382,289

(22) PCT Filed: Sep. 28, 2012
(86) PCT No.: PCT/BR2012/000374

§ 371 Date: Aug. 29, 2014

(30) Foreign Application Priority Data
Mar. 1, 2012 (BR) 10 2012 004682 2

(51) Int. Cl. A61L 27/46; B29C 44/02; A61L 27/56; A61L 27/58; A61L 27/54; B29C 44/34; B29C 67/20

(52) U.S. Cl. CPC A61L 27/46; B29C 44/3446; B29C 44/02; B29C 67/202; A61L 27/58; A61L 27/54; A61L 27/56
USPC 424/426; 264/49; 264/54; 424/602

(57) ABSTRACT

A bioresorbable and bioactive three-dimensional porous material made from bioresorbable polymers that can be combined with bioactive ceramics, producing a three-dimensional structure of interlinked pores containing additives capable of allowing the regeneration and formation of tissues, and a method for the production thereof is described.

### TECHNICAL FIELD

The present invention belongs to the field of tissue engineering and refers to a bioresorbable and bioactive three-dimensional porous material, such as a scaffold, that allows the regeneration and formation of tissues in mammals.

Furthermore the present invention refers to the process for the production thereof.

### BACKGROUND ART

The bone is a tissue subjected to continuous adaptation along a vertebrate's life, in order to obtain and preserve the size, shape and structural integrity of the skeleton, besides regulate mineral homeostasis.

The remodeling and formation processes constitute the basis for the development and maintenance of the skeletal system. When a lesion in the bone tissue occurs, the required repair consists of four main steps: fracture hematoma formation, cartilaginous callus formation, osseous callus formation and bone remodeling. The process takes at least four months and it depends on the intensity of the tissue injury.

Traumas or diseases can cause injuries or loss of tissue in the organism. In the event of osseous tissue loss, for instance, autogenous bone grafts, characterized by osteoconductivity, osteoinductivity and osteogenicity, are commonly used. Nevertheless this type of graft requires that bone transplant is from another region of the patient's body, generating problems with hematomas, pain, infections, absorption variations and risk of death, among others.

As an alternative to this procedure, homogeneous grafts from human tissue banks which present good absorption can be applied, although they present the risk of transmission of undetected infectious diseases.

In this context, tissue bioengineering has sought the creation of medical devices capable of repairing, restoring and regenerating tissues injured by diseases, injury or age, trying to combine the patient's benefit with the reduction of limiting side effects.

The use of porous scaffolds is one of the most effective methods for a three-dimensional tissue reconstruction, as it acts as a support for cell anchoring, resulting in guided tissue formation, that is, guided bone regeneration (GBR).

Within this context, this type of material requires some peculiarities and specific characteristics. First of all, biocompatibility is essential, since the material shall not provoke inflammatory response or immunogenicity or cytotoxicity.

Furthermore, the mechanic properties shall be enough to maintain material integrity and avoid its collapse, allowing structural stability of the regenerated tissue.

Degradation constitutes an important factor as well, as it can affect a lot of cellular processes, including cellular growth, tissue regeneration and compatibility response.

Ultimately, porosity and porous size shall be adequate to optimize the processes of cellular sedimentation, adhesion and growth, as well as the production of extracellular matrix, vascularization and tissue formation.

Some materials with the same aim of regenerating and creating new tissue in mammals already exist.

The prior art document PI 9602509-3, for instance, describes the application of bioresorbable materials and with controlled porosity, such as grafts or medications, for injury treatment. In this document, the pores are formed by freeze-drying, using different steps and solvents. However, the control of porous size and the density of obtained materials are crucial. Moreover, biopolymers of animal origin, such as collagen, for instance, are also used.

Collagen stimulates bone growth, as it is the main constituent of the extracellular matrix. However, its extraction is performed directly from biological tissues, although, its formulation is performed with the use of a recombinant protein, becoming relatively expensive and thermally instable, hampering its application in medical and pharmaceutical compositions.

The study by Jeong et al. (Jeong et al, J Biomater Sei Polymer Edn, 15, 645-660, 2004) describes the production of a porous material via salt leaching. Control of porous size can be reached by sieving the particles with a sieve having a specific porosity. A determinant and negative factor for what concerns this type of process is the long leaching time needed for the complete removal of the porosity forming agent, in this case represented by salt particles. The period required for the material formation in this type of procedure can last days.

The prior art document PI 0605628-8 discusses the manufacturing of porous aluminum matrices, with bioglass and hydroxyapatite infiltration, where porosity is obtained by using sucrose. Despite being an inert material, the use of aluminum requires a considerable time for tissue regeneration.

The use of inert metallic materials is quite common in orthopedic implants, for instance, in places that, in the absence of tissue, a replacement of the needed structure occurs. Various patent applications that describe materials for the use in prostheses and implants can be mentioned, including femoral stems (such as document PI 0802289-5) or dental implants (such as prior art document PI 9301263-2). Although they offer bone support, such materials should be changed over time, resulting in new surgical procedures.

Thus, the present invention describes a bioresorbable and bioactive three-dimensional porous material, as well as the obtaining method thereof.

The developed material is capable of changing both its polymeric and ceramic composition, when applicable, and also its pores, that can present different shapes, sizes, porosity (percentage of pores) and distribution.

Moreover, the material implant requires only one surgical procedure, reducing pain, postoperative complications and the risk of infections, as the material is completely reabsorbed by the organism.

Furthermore, this can be added to pharmacological agents, alone or in combination, with prophylactic purposes, for treatment or also for promoting tissue regeneration.

### SUMMARY OF THE INVENTION

The present invention refers to a bioresorbable and bioactive three-dimensional porous material, as well to the process for the production thereof. The material is made from polymers and, when applicable, bioactive ceramic and pharmacological agents.

### BRIEF DESCRIPTION OF DRAWINGS

FIG. 1 is an image obtained by scan electron microscopy, at a magnification of 3500 times, of the bioresorbable and bioactive three-dimensional porous material of the present invention.

### DETAILED DESCRIPTION OF THE INVENTION

The present invention consists of a bioresorbable and bioactive three-dimensional porous material, such as a scaffold, presenting adequate mechanical properties for different application sites, as it can be used as a platform or porous biomimetic support for bone defects or bone cavities, capable of regenerating and forming bone tissue in a reduced period of time if compared to the natural process and to its production process.

Such material comprises bioresorbable polymers, offering a three-dimensional physical structure and mechanical characteristics required for the support.

The base polymeric matrix is comprised of polymers selected from lactide monomers and/or homopolymers in all the possible isomeric variants, such as D-lactide, L-lactide, DL-lactide; ε-caprolactone monomer and/or homopolymer; glicolyde monomer and/or homopolymer; poly(hydroxyalkanoate); polyesters and polyamides derived from aliphatic dicarboxylic acids and aliphatic hydroxyacids or aliphatic amino acids; poly(carprolactam); poly(dioxanone); poly(trimethylene carbonate); poly(urethane)s; as well as copolyesters, copolyamides and copolyester-amide derived therefrom and mixtures thereof, that are added in monomeric ratio varying from 0.1% to 99.9%, for each constituent, in the total composition for the formation of the base polymeric matrix.

The base polymeric matrix can be supplemented with hydrophilic polymers and, also, bioactive additives, such as ceramic particles, serving as cell signaling and consequent stimulus for formation, growth and bone tissue regeneration.

Hydrophilic polymers are added in mass ratios varying from 10 to 75% of the total mass of the base polymer matrix and they are selected from polyacrylics, amine functional polymers, polyethers, polystyrenes, poly(vinyl acids), poly(vinyl alcohols), (poly)vinylpyrrolidone, sodium polystyrene maleate, natural origin polymers (such as gelatin, starch, modified cellulose and chitin) and, preferably, ethylene polyoxides and polyethylenes glycols.

Ceramic particles are comprised of calcium phosphate, and are selected among the group comprising tetracalcium phosphate [TeCP, Ca4O(PO4)2], hydroxyapatite [HA, Cai0(PO4)6(OH)2] and its size variation in the manometric scale (nanoHA), amorphous calcium phosphate [ACP, Ca3(PO4)2.nH2), tricalcium phosphate (α, α′, β, γ) [TCP, Ca3(PO4)2], octacalcium phosphate [OCP, Ca8H2(PO4)6-5H2O], calcium monohydrogen phosphate dihydrate [DCPD, CaHPO4.2H2O], calcium monohydrogen phosphate [DCP, Ca2P2O7.2H2O], heptacalcium phosphate [HCP, Ca7(P5O16)2], tetracalcium dihydrogen phosphate [TDHP, Ca4H2P6O20], monocalcium monohydrogen phosphate [MCPM, Ca(H2PO4)2.H2O] and calcium metaphosphate (α, β, γ) [CMP, Ca(P03)2] and are added in a range that can vary from 0.01 to 20.0%.

The material can be also supplemented with medicines, alone or in combination, with prophylactic purposes, for treatment or also for promoting tissue regeneration.

The addition of antibiotics and anti-inflammatories to the bioresorbable and bioactive three-dimensional porous material of the present invention allows a local and controlled administration of the medicine, increasing its efficacy and reducing possible systemic toxic effects

Similarly, in case of tumors, it is possible to direct the chemotherapy treatment without major damage to the individual.

Among the most used antitumorals for bone tumor control, cisplatin, doxorubicin, ifosfamide, methotrexan, cyclofosfamide, etoposide and irinotecan, can be selected.

The concentration of these medicines can vary from 0.1% to 50% by mass. Preferably, the antitumoral agents are added in a concentration from 2% to 30% by mass.

By considering bone regeneration or tissue regeneration as a whole (as it occurs in soft and cartilaginous tissues), the bioresorbable and bioactive three-dimensional porous material can be supplemented with an antibiotic, selected among the group consisting of: macrolides (erythromycin and azithromycin); tetracyclines (tetracycline, doxycycline and minocycline); β-lactams (penicillin, cephalosporin, carbapenems and clavunates), glycopeptides (vancomycin); aminoglycosides (tobramycin, streptomycin, gentamicin) and licosamides (clyndamicin).

The concentration of these medicines can vary from 0.1% to 50% by mass. Preferably, antitumoral agents are added in a concentration from 1% to 15% by mass.

For guided bone regeneration, the use of steroidal and non-steroidal anti-inflammatories can impair bone regeneration process. Thus, the use of alternative medicines, such as hypolipidemic agents, is recommended.

Among them, the class of statins, especially, simvastatin, atorvastatin, lovastatin, fluvastatin and pravastatin, that can be added to the bioresorbable and bioactive three-dimensional porous material in the concentration ranging from 0.01% to 50% by mass. Preferably, statins are added in a concentration from 0.5% to 10% by mass.

Considering the use of the bioresorbable and bioactive three-dimensional porous material for other purposes, steroidal anti-inflammatories, especially those selected among dexamethasone, hydrocortisone, betamethasone, prednisolone, methylprednisolone, cortisone and corticosterone, can be included in a concentration that can vary from 0.1%% to 50% by mass, preferably from 1% to 20% by mass.

It is also possible to include non-steroidal anti-inflammatories, especially COX inhibitors, that are selected from the group consisting of salycilates (acetylsalicylic acid); indol- and indoleacetic acids (indomethacin, sulindac and etodolac), heteroarylacetic acids (diclofenac, ketorolac, aceclofenac and tolmetin); arylpropionic acids (ibuprofen, naproxen, flurbiprofen, ketoprofen, loxoprofen and oxaprozin); anthranilic acids (mefenamic acid and meciofenamic acid); enolic acids (piroxicam, tenoxicam and meloxicam); alcanones (nabumetone); coxibs (rofecoxib, celecoxib and etoricoxib); para-aminophenol (paracetamol) and sulfonanilides (nimesulide) in a concentration that can vary from 0.1% to 50% by mass, preferably, from 1% to 20% by mass.

Some properties are desirable and essential in the material, such as degradation control and reabsorbance of scaffold components, adequate mechanic resistance and efficient porosity to allow vascularization in the whole structure of the material.

These characteristics allow cell migration to the inner part of the material and facilitate cellular growth for the formation of specific tissue.

Material Formation Process:

The bioresorbable and bioactive three-dimensional porous material of the present invention is obtained through the process comprising the following steps:

Step 1—Polymers Dissolution for Preparation of the Base Polymeric Matrix

In order to obtain the material of the present invention, combination of polymers of natural or synthetic origin are used, that can undergo degradation to non-toxic hydroacids through hydrolytic and/or enzymatic processes.

Said polymers are selected according to their biocompatibility and biodegradability and are used in monomeric ratios varying from 0.1% to 99.9%, for each component, in the total composition for the formation of the base polymeric matrix. Polymers are selected from the group consisting of: lactide monomers and/or homopolymers in all the possible isomeric variants, such as D-lactide, L-lactide, DL-lactide; ε-caprolactone monomers and/or homopolymers; glicolyde monomers and/or homopolymers; poly(hydroxyalkanoate); polyesters and polyamides derived from aliphatic dicarboxylic acids and aliphatic hydroxyacids or aliphatic amino acids; poly(carprolactam); poly(trimethylene carbonate); poly(dioxanone); poly(urethanes); as well as copolyesters, copolyamides and copolyester-amide derived therefrom and mixtures thereof.

A preferred ratio for the polymeric matrix formation process comprises lactide:caprolactone (LL:CL) from 1:99 to 99:1, preferably from 30:70 to 70:30 m/m.

In the first step of the material formation process, polymers are dissolved for the formation of the base polymeric matrix.

For polymers dissolution, polar organic solvents of medium or small polarity are used, such as halogenated solvents, that is, solvents containing chlorine, fluorine, bromine or iodine atoms (such as chloroform, dichloromethane, carbon tetrachloride, trichloroethane and bromoform); 1-4 dioxane; propylene carbonate; acetone; methyl acetate; tetrahydrofuran; pyridine and formic acid (98%), among others. Preferably chlorinated solvents, such as chloroform and dichloromethane, are used.

Polymers dissolution is performed at room temperature and under stirring from low to high intensity, about 50 to 500 rpm, in a magnetic stirrer.

First, the constituent polymers of the base polymeric matrix, that is lactide, glycolide and/or ε-caprolactone-based homopolymers or copolymers, are dissolved in the chlorinated solvent.

In preferred embodiments, the base polymeric matrix is comprised of lactide, glycolide and/or caprolactone-based copolymers supplemented with a hydrophilic polymer.

When applicable, the hydrophilic polymer is added to the dispersion of polymers constituting the base polymeric matrix, in mass ratios, varying from 10 to 75%, with respect to the total mass of the base polymeric matrix, and are selected from the group consisting of poly(acrylics), amine functional polymers, poly(ethers), poly(styrenes), poly(vinyl acids), poly(vinyl alcohols), poly(vinylpyrrolidone), poly(styrene-sodium maleate), natural origin polymers (such as gelatin, starch, modified cellulose and chitin) and, preferably, polyethylene oxides) and poly(ethylene glycols).

Furthermore, when applicable, calcium phosphate-based bioactive ceramics can be added in a range from 0.01 to 20%. Ceramics are biocompatible and osteoinductive, and, besides not constituting a risk of transmissible diseases, they are toxicity-free and can cause minimal immune reaction.

Ceramic particles are selected from the group consisting of tetracalcium phosphate [TeCP, Ca₄O(PO₄)₂], hydroxyapatite [HA, Ca₁₀(PO₄)₆(OH)₂] and its size variation in the manometric scale (nanoHA), amorphous calcium phosphate [ACP, Ca₃(PO₄)₂.nH₂), tricalcium phosphate (α, α′, β, γ) [TCP, Ca₃(PO₄)₂], octacalcium phosphate [OCP, Ca₈H₂(PO₄)₆-5H₂O], dihydrated calcium monohydrogen phosphate [DCPD, CaHPO₄.2H₂O], calcium monohydrogen phosphate [DCP, Ca₂P₂O₇.2H₂O)], heptacalcium phosphate [HCP, Ca₇(P₅O₁₆)₂], tetracalcium dihydrogen phosphate [TDHP, Ca₄H₂P₆O₂₀], monohydrated monocalcium phosphate [MCPM, Ca(H₂PO₄)2.H₂O] and calcium metaphosphate (α, β, γ) [CMP, Ca(PO₃)₂].

When there is addition of bioactive additives, such as ceramic particles, these are added in the first step of dissolution of the polymers constituting the base polymeric matrix.

The addition of the hydrophilic polymer, or the mixture of hydrophilic polymers, is performed when the dispersion of base polymeric matrix is homogeneous with respect to the ceramic particles.

In this step the addition of antitumoral agents, antibiotics and anti-inflammatories, when applicable, is also taken into account, in an adequate concentration according to the nature of the agent, respecting the toxicity limits and the minimum effective concentration.

Antitumorals are selected from the group of cisplatin, doxorubicin, ifosfamide, methotrexan, cyclofosfamide, etoposide and irinotecan, and are added in a concentration that may vary from 0.1% to 50% by mass, preferably from 2% to 30% by mass.

Antibiotics are selected from the group consisting of: macrolides (erythromycin and azithromycin); tetracyclines (tetracycline, doxycycline and minocycline); β-lactams (penicillin, cephalosporin, carbapenems and clavunates), glycopeptides (vancomycin); aminoglycosides (tobramycin, streptomycin, gentamicin) and licosamides (clyndamicin) and are added in a concentration that may vary from 0.1% to 50% by mass, preferably, from 1% to 15% by mass.

Among hypolipidemic agents, statins selected from the group comprising simvastatin, atorvastatin, lovastatin, fluvastatin and pravastatin, can be added in a concentration from 0.01% to 50% by mass, preferably, in a concentration from 0.5% to 10% by mass.

Furthermore steroidal anti-inflammatories selected from the group of dexamethasone, hydrocortisone, betamethasone, prednisolone, methylprednisolone, cortisone and corticosterone can be added, and non-steroidal anti-inflammatories selected from the group consisting of salycilates (acetylsalicylic acid); indol- and indoleacetic acids (indomethacin, sulindac and etodolac), hetero aryl-acetic acids (diclofenac, ketorolac, aceclofenac and tolmetin); arylpropionic acids (ibuprofen, naproxen, flurbiprofen, ketoprofen, loxoprofen and oxaprozin); anthranilic acids (mefenamic acid and meclofenamic acid); enolic acids (piroxicam, tenoxicam and meloxicam); alcanones (nabumetone); coxibs (rofecoxib, celecoxib and etoricoxib); para-aminophenol (paracetamol) and sulfonanilides (nimesulide), in a concentration that may vary from 0.1% to 50% by mass, preferably, from 1% to 20% by mass.

Step 2—Addition of Porosity-Forming Agent:

To cause the formation of interconnected pores, with an average diameter ranging from 50 to 220 μm, porosity-forming agents are used.

Such agents can be: sugar particles, both monosaccharides (fructose and glucose) or disaccharides (sucrose; inorganic salts particles such as sodium chloride and ammonium carbonate; frozen or effervescent particles/droplets of solvent insoluble in the dispersion of polymeric matrix, such as n-hexane, methanol, ethanol, isopropanol; gas exhibiting effervescent reaction, by using ammonium bicarbonate, for example, or gas foam may be used.

The porosity-forming agent shall be added in varying ratios in respect to the mass of the dispersion of polymeric matrix, preferably in equal or higher ratios, that may vary from 1:1 to 20:1 (m/m), so as to provide a porosity of up to 90% in the material.

The addition of the porosity-forming agent to the dispersion of the polymeric matrix can be performed in any phase of the dissolution, preferably, after complete homogenization of the dispersion.

Step 3—Mixture Shaping:

The preparation of the dispersion for the leaching process, also called shaping, is the step of the process where the polymeric mixture, with the addition of the porosity-forming agent, is homogenized until it reaches a firm and adequate consistency for modeling and consequent leaching.

In this step, the almost complete elimination of the organic solvent material occurs. Thereunto, the use of temperature aids the process of solvent evaporation and the consequent reduction in the process time.

Dissolution temperature shall be higher than the boiling point of the used solvent, so that the shaping time is reduced with respect to the required time at room temperature.

By using a temperature higher than the solvent boiling point, the shaping or preparation time of the mixture takes about 10 minutes.

In this process, stirring is constant from low to medium intensity, ranging from 50 to 500 rpm, so that the resultant mixture is homogenous.

Step 4—Removal of Porosity-Forming Agent

The removal step depends on the porosity-forming agent used in the material.

Among the processes that can be used for the removal of the porosity-forming agent, leaching, porogen melting, sintering, pressure drop and porogen dissolution can be used.

After removal of the porosity-forming agent, the bioresorbable three-dimensional porous material or scaffold is obtained.

Step 5—Material Drying:

Material drying can be optimized by using temperature.

Drying temperature shall be lower than the glass transition temperature of the used polymer or than PEG melting temperature, preferably under reduced pressure, or under vacuum. Thus, the material can be heated at a suitable temperature for approximately two hours, under vacuum, and subsequently kept under vacuum at room temperature until its later use.

Depending on the monomeric composition, glass transition temperature comprises the interval between −40.0° C. and 60.0° C.

When a hydrophilic polymer is used, the presence of water (especially in the form of moisture) shall be avoided, due to the hydrophilicity of this component.

The drying procedure is the same, however, it shall be considered that the temperature of the drying process is also lower than the melting temperature or glass transition temperature of the used hydrophilic polymer.

In a preferred process, the drying step can be performed only under reduced pressure, or under vacuum, at room temperature, which is enough to keep the material free from moisture.

Material drying can be performed with the aid of nitrogen gas of 99.99% purity grade with subsequent storing under vacuum and at room temperature.

Step 6—Material Sterilization:

After dried and packed, the obtained material shall be sterilized.

Among the possible sterilization methods, treatment with ethylene oxide or gamma irradiation can be employed, among others.

### First Specific Example of the Invention

A 12.5% poly(L-lactide-co-ε-caprolactone) (PLCL) dispersion 70:30 (L-lactide: ε-caprolactone m/m) containing 3.75% ceramic (hydroxyapatite: β-tricalcium phosphate 1:1 m/m) in dichloromethane is prepared by moderate stirring of about 400 rmp, in a magnetic stirrer, at room temperature until complete homogenization (approximately 5 minutes).

To this mixture 4,000 g·mol″¹ PEG is added until the ratio of 2:1 PLCLPEG m/m is reached, keeping constant stirring until complete homogenization (about 2 minutes).

After dispersion of the polymeric matrix in dichloromethane, sucrose is added as a porogenic agent, in the granulometric range of 50-125 μm, in a ratio of 3:1 with respect to the used PLCL mass.

The polymeric dispersion containing sucrose is led to a temperature higher than dichloromethane boiling point, in this case, 70° C. The mixture is prepared and shaped under constant stirring, at a moderate level, so that it is possible to handle and transfer it to an hollow Teflon mold.

After preparation of the mixture in the mold, it follows the leaching process, for the removal of porogenic agent particles and consequent pore formation.

Sucrose removal was obtained by using water, in a convenient period of time for total removal, preferably between 5 and 15 minutes.

The amount of water used was 1000 times higher than sucrose solubility in water, in order to ensure complete removal of sugar particles from the material composition. Leaching process was performed in duplicate, with water exchange.

Drying was performed in 2 steps: (i) washing of the material in ice-cold analytical grade (around 0° C.) ethylic alcohol and removing of the excess of alcohol and (ii) storing in an atmosphere under reduced pressure (vacuum) for 12 hours.

### Second Specific Example of the Invention

Until the step of shaping the mixture into the mold, the process executed in example 2 was identical to that described in example 1.

Leaching was performed in a microwave equipment, more specifically, a conventional microwave oven.

Thereunto, water is pre-heated in a microwave oven until boiling, preferably, in a period comprising 1 to 5 minutes.

After the water reaches its boiling temperature, the material is immersed therein and the leaching process begins, executed in duplicate, again under microwave, for time needed for complete removal of the porous-forming agent, preferably between 5 and 15 minutes.

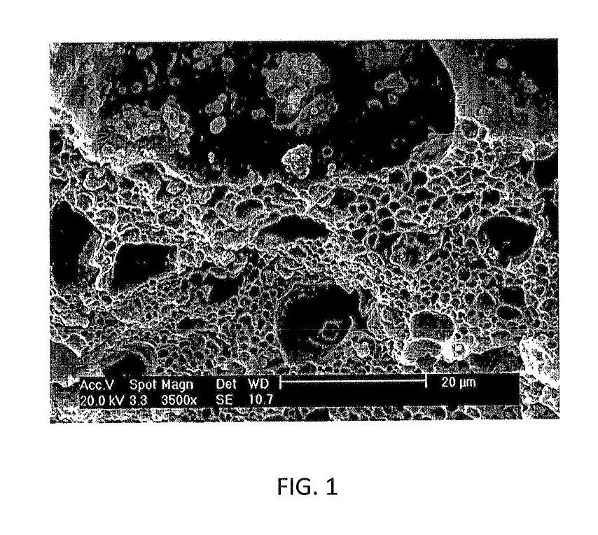

Illustrative images of the bioresorbable and bioactive three-dimensional porous material made of poly(L-lactide-co-ε-caprolactone) 70:30, by the process of the present invention, are shown in FIGS. 1, 2A and 2B.

FIG. 1 is an image of the porous material obtained by scan electron microscopy at a magnification of 3500 times, wherein it is possible to observe the connectivity between the pores in a three-dimensional structure, in appropriate size to allow vascularization and cell anchoring.

FIG. 2A shows the material prepared with poly(L-lactide-co-ε-caprolactone)—PLCL in 40:60 L-lactide-co-ε-caprolactone monomeric ratios and 4000 g·mol″¹ PEG, prepared with sucrose as the porosity-forming agent.

FIG. 2B shows the material prepared with poly(L-lactide-co-ε-caprolactone)—PLCL in 70:30 L-lactide-co-ε-caprolactone monomeric ratios and 4000 g·mol″¹ PEG, prepared with sucrose as the porosity-forming agent.

As described, the bioresorbable and bioactive three-dimensional porous material provides a support that allows the support and filling of the deficient region of the tissue, providing a structure capable of allowing vascularization for cells adherence and subsequent differentiation and growth thereof for the required formation or regeneration of tissue.

Although the preferred version of the invention has been illustrated and described, it should be understood that the invention is not limited. Various modifications, changes, variations, substitutions and equivalents may occur without departing from the scope of the invention.

1. Bioresorbable and bioactive three-dimensional porous material characterized in that it comprises a base polymeric matrix comprised of combinations of polymers of natural or synthetic origin, that are selected from the group consisting of: lactide monomers and/or homopolymers in all the possible isomeric variants, such as D-lactide, L-lactide, DL-lactide; ε-caprolactone monomers and/or homopolymers; glicolyde monomers and/or homopolymers; polyesters and polyamides derived from aliphatic dicarboxylic acids and aliphatic hydroxyacids or aliphatic amino acids; poly(hydroxyalkanoate); poly(carprolactam); poly(trimethylene carbonate); poly(urethanes); as well as copolyesters, copolyamides and copolyester-amide derived therefrom, and/or mixtures thereof, wherein polymers are in monomeric ratios varying from 0.1% to 99.9% of the total composition of the base polymeric matrix.

2. Material, according to claim 1, characterized in that it also comprises ceramic particles in mass ratios varying from 0.01 to 20.0% with respect to the total mass of the polymeric matrix.

3. Material, according to claim 1, characterized in that it also comprises a hydrophilic polymer in mass ratios varying from 10 to 75% with respect to the total mass of the base polymeric matrix.

4. Material, according to claim 1, characterized in that it also comprises antitumoral agents, antibiotics, hypolipidemics and/or anti-inflammatories or combinations thereof.

5. Material, according to claim 1, characterized in that the base polymeric matrix is comprised of copolymers comprised of lactide, glycolide and/or caprolactone.

6. Material, according to claim 5, characterized in that the base polymeric matrix is comprised of lactide:caprolactone from 1:99 to 99:1 by mass.

7. Material, according to claim 6, characterized in that the base polymeric matrix is comprised of lactide:caprolactone from 30:70 to 70:30 by mass.

8. Material, according to claim 2, characterized in that ceramic particles are selected from the group of calcium phosphates.

9. Material, according to claim 8, characterized in that calcium phosphates are selected from the group consisting of: tetracalcium phosphate [TeCP, Ca₄O(PO₄)₂], hydroxyapatite [HA, Ca₁₀(PO₄)₆(OH)₂] and its size variation in the manometric scale (nanoHA), amorphous calcium phosphate [ACP, Ca₃(PO₄)₂.nH₂), tricalcium phosphate (α, α′, β, γ) [TCP, Ca₃(PO₄)2], octacalcium phosphate [OCP, Ca₈H₂(PO₄)₆-5H₂O], calcium monohydrogen phosphate dihydrate [DCPD, CaHPO₄.2H₂O], calcium monohydrogen phosphate [DCP, Ca₂P₂O₇.2H₂O], heptacalcium phosphate [HCP, Ca₇(P₅O₁₆)₂], tetracalcium dihydrogen phosphate [TDHP, Ca₄H₂P₆O₂₀], monocalcium monohydrogen phosphate [MCPM, Ca(H₂PO₄)2.H₂O] and calcium metaphosphate (α, β, γ) [CMP, Ca(PO₃)₂], nanoHA, HA and β-TCP.

10. Material, according to claim 3, characterized in that the hydrophilic polymer is selected from the group consisting of poly(acrylics), amine functional polymers, poly(ethers), poly(styrenes), poly(vinyl acids), poly(vinyl alcohols), (poly)vinylpyrrolidone, poly(styrene-sodium maleate), natural origin polymers (such as gelatin, starch, modified cellulose and chitin), poly(ethylene oxides) or poly(ethylene glycols).

11. Material, according to claim 4, characterized in that antitumorals are selected from the group consisting of cisplatin, doxorubicin, ifosfamide, methotrexan, cyclofosfamide, etoposide and irinotecan, and are added in a concentration varying from 0.1% to 50% by mass, preferably from 2% to 30% by mass.

12. Material, according to claim 4, characterized in that antibiotics are selected from the group consisting of macrolides (erythromycin and azithromycin); tetracyclines (tetracycline, doxycycline and minocycline); β-lactams (penicillin, cephalosporin, carbapenems and clavunates), glycopeptides (vancomycin); aminoglycosides (tobramycin, streptomycin, gentamicin) and licosamides (clyndamicin) and are added in a concentration varying from 0.1% to 50% by mass, preferably, from 1% to 15% by mass.

13. Material, according to claim 4, characterized in that hypolipidemics are selected from the group of statins, especially simvastatin, atorvastatin, lovastatin, fluvastatin and pravastatin, and are added in a concentration from 0.01% to 50% by mass, preferably, in a concentration from 0.5% to 10% by mass.

14. Material, according to claim 4, characterized in that anti-inflammatories can be steroidal and non-steroidal and are added in a concentration varying from 0.1% to 50% by mass, preferably, from 1% to 20% by mass.

15. Material, according to claim 14, characterized in that steroidal anti-inflammatories are selected from the group consisting of dexamethasone, hydrocortisone, betamethasone, prednisolone, methylprednisolone, cortisone and corticosterone.

16. Material, according to claim 14, characterized in that non-steroidal anti-inflammatories are selected from the group consisting of salycilates (acetylsalicylic acid); indol- and indoleacetic acids (indomethacin, sulindac and etodolac), heteroaryl-acetic acids (diclofenac, ketorolac, aceclofenac and tolmetin); arylpropionic acids (ibuprofen, naproxen, flurbiprofen, ketoprofen, loxoprofen and oxaprozin); anthranilic acids (mefenamic acid and meclofenamic acid); enolic acids (piroxicam, tenoxicam and meloxicam); alcanones (nabumetone); coxibs (rofecoxib, celecoxib and etoricoxib); para-aminophenol (paracetamol) and sulfonanilides (nimesulide).

17. Process to obtain the bioresorbable three-dimensional porous material as defined in claim 1 characterized in that it comprises the steps of:
a. dissolving polymer in order to prepare the base polymeric matrix;
b. adding porosity-forming agent;
c. shaping of the mixture;
d. removing porosity-forming agent;
e. drying of the material;
f. sterilizing of the material.

18. Process, according to claim 17, characterized in that, in step (a), polymers are dissolved in a polar organic solvent with medium or small polarity, at room temperature and under stirring from 50 to 500 rpm in magnetic stirrer.

19. Process, according to claim 18, characterized in that polymers are selected from the group consisting of: lactide monomers in all the possible isomeric variants, such as poly(D-lactide), poly(L-lactide), poly(DL-lactide); caprolactone monomers in all the possible molecular structural variants, such as poly(caprolactone) and s-caprolactone; glicolyde monomers in all the possible molecular structural variants, such as poly(glicolyde); polyesters and polyamides derived from aliphatic dicarboxylic acids and aliphatic hydroxyacids or aliphatic amino acids; poly(hydroxyalkanoate); poly(carprolactam); poly(trimethylene carbonate); poly(urethanes); as well as copolyesters, copolyamides and copolyester-amide derived therefrom, wherein polymers are present in monomeric ratios varying from 0.1% to 99.9% of total composition of the base polymeric matrix.

20. Process, according to claim 18, characterized in that the polar organic solvent with medium or small polarity is a halogenated solvent (such as chloroform, dichloromethane, carbon tetrachloride, trichloroethane and bromoform); 1-4 dioxane; propylene carbonate; acetone; methyl acetate; tetrahydrofuran; pyridine and formic acid (98%).

21. Process, according to claim 18, characterized in that the polar organic solvent with medium or small polarity is a chlorinated solvent.

22. Process, according to claim 21, characterized in that the polar organic solvent with medium or small polarity is chloroform or dichloromethane.

23. Process, according to claim 17, characterized in that further comprises the addition of ceramic particles after dissolution of the polymers constituting the base polymeric matrix.

24. Process, according to claim 23, characterized in that ceramic particles are calcium phosphates selected from the group consisting of tetracalcium phosphate [TeCP, Ca₄O(PO₄)₂], hydroxyapatite [HA, Ca₁₀(PO₄)₆(OH)₂] and its size variation in the manometric scale (nanoHA), amorphous calcium phosphate [ACP, Ca₃(PO₄)₂.nH₂), tricalcium phosphate (α, α′, β, γ) [TCP, Ca₃(PO₄)₂], octacalcium phosphate [OCP, Ca₈H₂(PO₄)₆-5H₂O], calcium monohydrogen phosphate dihydrate [DCPD, CaHPO₄.2H₂O], calcium monohydrogen phosphate [DCP, Ca₂P₂O₇.2H₂O], heptacalcium phosphate [HCP, Ca₇(P₅O₁₆)₂], tetracalcium dihydrogen phosphate [TDHP, Ca₄H₂P₆O₂₀], monocalcium monohydrogen phosphate [MCPM, Ca(H₂PO₄)2.H₂O] and calcium metaphosphate (α, β, γ) [CMP, Ca(PO₃)₂], nanoHA, HA β-TCB present in mass ratios varying from 0.01 to 20.0% with respect to the total mass of the polymeric matrix.

25. Process, according to claim 17, characterized in that it further involves the addition of a hydrophilic polymer after homogenization of ceramic particles in the base polymeric matrix.

26. Process, according to claim 25, characterized in that the hydrophilic polymer is selected from the group consisting of polyacrylics, amine functional polymers, poly(ethers), poly(styrenes), poly(vinyl acids), poly(vinyl alcohols), poly(vinylpyrrolidones), poly(styrene-sodium maleate), natural origin polymers (such as gelatin, starch, modified cellulose and chitin), poly(ethylene oxides) and poly(ethylenes glycols), wherein the hydrophilic polymer is presented in mass ratios varying from 10 to 75% with respect to the total mass of the base polymeric matrix.

27. Process, according to claim 17, characterized in that it further involves the addition of antitumoral agents, antibiotics, hypolipidemics and/or anti-inflammatories, or combinations thereof, after homogenization of ceramic particles in the base polymeric matrix

28. Material, according to claim 27, characterized in the antitumorals are selected from the group consisting of cisplatin, doxorubicin, ifosfamide, methotrexan, cyclofosfamide, etoposide and irinotecan, and are added in a concentration varying from 0.1% to 50% by mass, preferably from 2% to 30% by mass.

29. Material, according to claim 27, characterized in the antitumorals are selected from the group consisting of macrolides (erythromycin and azithromycin); tetracyclines (tetracycline, doxycycline and minocycline); β-lactams (penicillin, cephalosporin, carbapenems and clavunates), glycopeptides (vancomycin); aminoglycosides (tobramycin, streptomycin, gentamicin) and licosamides (clyndamicin) and are added in a concentration varying from 0.1% to 50% by mass, preferably from 1% to 15% by mass.

30. Material, according to claim 27, characterized in that hypolipidemics are selected from the group of statins, especially simvastatin, atorvastatin, lovastatin, fluvastatin and pravastatin, and are added in a concentration from 0.01% to 50% by mass, preferably, in a concentration from 0.5% to 10% by mass.

31. Material, according to claim 27, characterized in that anti-inflammatories can be steroaidal or non-steroidal and are added in a concentration varying from 0.1% to 50% by mass, preferably, from 1% to 20% by mass.

32. Material, according to claim 31, characterized in that steroidal anti-inflammatories are selected from the group consisting of dexamethasone, hydrocortisone, betamethasone, prednisolone, methylprednisolone, cortisone and corticosterone.

33. Material, according to claim 31, characterized in that non-steroidal anti-inflammatories are selected from the group consisting of salycilates (acetylsalicylic acid); indol- and indoleacetic acids (indomethacin, sulindac and etodolac), heteroarylacetic acids (diclofenac, ketorolac, aceclofenac and tolmetin); arylpropionic acids (ibuprofen, naproxen, flurbiprofen, ketoprofen, loxoprofen and oxaprozin); anthranilic acids (mefenamic acid and meclofenamic acid); enolic acid (piroxicam, tenoxicam and meloxicam); alcanones (nabumetone); coxibs (rofecoxib, celecoxib and etoricoxib); para-aminophenol (paracetamol) and sulfonanilides (nimesulide).

34. Process, according to claim 17, characterized in that, in step (b), the porosity-forming agent is added in ratios varying from 1:1 to 20:1 by mass with respect to the dispersion mass of the polymeric matrix, and it is selected from the group consisting of: sugar particles (monosaccharides, such as fructose and glucose, or disaccharides, such as sucrose); particles of inorganic salts, such as sodium chloride and ammonium carbonate; frozen or effervescent particles and/or droplets of solvent insoluble in the polymeric matrix, such as n-hexane, methanol, ethanol, isopropanol; gas exhibiting effervescent reaction, by using ammonium bicarbonate or gas foam.

35. Process, according to claim 17, characterized in that, in step (c), the polymeric dispersion is homogenized under stirring ranging from 50 to 500 rpm until a firm consistency is reached.

36. Process, according to claim 35, characterized in that, in step (c), shaping is optimized by the use of temperature, where it is higher than the boiling point of the used solvent.

37. Process, according to claim 17, characterized in that, in step (d), the removal of the porosity-forming agent occurs by means of leaching, porogen melting, sintering, pressure drop and porogen dissolution.

38. Process, according to claim 17, characterized in that, in step (e), drying of the material is optimized by using temperature, wherein it is lower than the glass transition temperature of the used polymer.

39. Process, according to claim 17, characterized in that, in step (e), drying can be performed under reduced pressure or under vacuum.

40. Process, according to claim 38, characterized in that, in step(e), drying can also be performed with the aid of 99.99% pure nitrogen gas.

41. Process, according to claim 17, characterized in that sterilization is performed by treatment with ethylene oxide or gamma irradiation.
